(19) United States
(12) Patent Application Publication
(10) Pub. No.: US 2008/0033459 A1
(43) Pub. Date: Feb. 7, 2008

(54) Suturing apparatus for closing tissue defects

(75) Inventors: Bilal Shafi, Palo Alto, CA (US); Gabriel Sanchez, Belen, NM (US); Carlos Mery, Chestnut Hill, MA (US); Gary Binyamin, Palo Alto, CA (US); Jessica Connor, Palo Alto, CA (US)

Correspondence Address: Bilal Shafi, 213 Charles Marx Way, Palo Alto, CA, 94304, US

(73) Assignee: SurgSolutions, LLP, Palo Alto, CA (US)

(21) Appl. No.: 11/833,963

(22) Filed: Aug. 3, 2007

Related U.S. Application Data
(60) Provisional application No. 60/835,265, filed on Aug. 3, 2006.

(51) Int. Cl. A61B 17/04

(52) U.S. Cl. 606/144

(57) ABSTRACT

A device is provided to rapidly, safely, efficiently and effectively close tissue defects created to access operative sites in a patients body. The device comprises an elongated body with its features in profile to allow use through conduits such as trocars which can be removed over the device. The device has diametrically opposed pivoting extensions which provide a target for flexible needles to attach to suture and provide protection for all vital structures in the body. Once the pivoting extensions are past the innermost layer adjacent to the tissue defect the flexible needles are advanced through the tissue to obtain an adequate and proper amount of tissue to allow for a strong closure. The device leaves a suture behind which can then be tied to close the tissue defect.

### CROSS REFERENCE TO RELATED APPLICATIONS

This application claims the benefit of U.S. Provisional Application Ser. No. 60/A35,265 filed on Aug. 3, 2006 and is hereby incorporated as reference in its entirety.

### BACKGROUND OF THE INVENTION

1. Field of Invention

This invention relates generally to suturing devices for tissue defects, and more specifically to an improved system to facilitate the closure of the small tissue defects created by physicians for access to operative sites.

2. Background

The field of surgery was revolutionized in 1987 when a cholecystectomy was performed through very small incisions using a camera by Dr. Phillippe Mouret. Soon after that first procedure Dr. Francois Dubois introduced the concept of the trocar to facilitate access to the abdominal cavity allowing for minimal access surgery. Interest in the field and the number of procedures performed laparoscopically spread like wildfire, providing significant advantages to the patient over open procedures. Patients had shorter hospital stays, less pain, and smaller less disfiguring scars, with the result that patients were requesting their procedures to be done laparoscopically. With increasing demand from the patients, and interest from physicians, numerous innovations were made to improve the procedure and address the new specific complications associated with minimal access surgery.

A key component to performing laparoscopic surgery is obtaining access to the operating space by the placement of a trocar. One of the most difficult, time-consuming, and sometimes unreliable parts of the case is closing these incisions, especially in obese patients. This is mainly because these incisions are very small and the layer that needs to be closed (fascia) rests deep underneath the skin and fat tissue of the abdominal wall. The goal is to close these port sites which may range in sizes from 5 mm to 25 mm without enlarging the skin incision, without injuring the intracorporeal contents (i.e. the bowel in the abdominal cavity), and ensuring good fascial closure in a timely manner.

Fascial closure is instrumental in preventing the possibility of life threatening complications. Port or trocar site hernia is a recognized complication of laparoscopy with an incidence ranging from 0.23% to 3.10% depending on the study. This incidence only represents those patients who seek medical attention for symptomatic hernias. Patients with trocar site hernias present with symptoms ranging from unsightly bulges to the more life threatening incarceration of bowel. Typically when patients present with signs of an obstruction due to hernias, they must be repaired surgically and may require a small bowel resection. In rare cases, obese patients may develop a Richter's hernia where only parts of the small bowel are incarcerated in the hernia. These patients do not present with the classical symptoms of obstruction often leading to a missed or delayed diagnosis resulting in increased morbidity.

The pathogenesis of a trocar site hernia is multifactorial including poor closure technique, poor visualization, patient co morbidities including wound infection, diabetes, obesity, poor nutrition, or merely the decision to not close the trocar site. Trocar sites tend to be difficult to close, especially in obese patients, due to the fact that the depth of the incision does not allow proper visualization of the fascia in these incisions. The lack of visualization and the degree of frustration either leads the physician to decide not to close the port site or to attempt a suboptimal closure which may take more time than what the physician would prefer to spend (sometimes up to 20 minutes). Current techniques of closure can also be dangerous since intra-abdominal organs may be injured upon entry to the abdominal cavity or the bite size of fascia at each side of the wound may be too small, leading to a suboptimal closure and potential breakdown of the closure. Therefore there is a need for an improved, automatic, reliable, safe, and quick method to close trocar or port sites following laparoscopic surgery.

### Current Treatments and Prior Art

There is an ongoing debate concerning which size trocar sites should be closed and which can be left open. The majority of clinicians believe any port site 10 mm or larger made in the midline linea alba or lateral ports below the arcuate line should be closed in adults. In addition, most clinicians agree that any port 5 mm or larger should be closed in children. This decision has been made with reports of hernias occurring in people with port sites smaller than 10 mm. Some clinicians have suggested closing smaller trocar sites in adults if there was significant manipulation of the site during the surgery. The reality is that by not closing these sites, patients are being placed at an increased risk of a re-operation and possibly a life threatening problem.

Currently, clinicians have limited options for closing laparoscopic trocar sites. Most of the devices currently on the market are either more complicated than the standard techniques or place the patient and or physician at risk for injury. In the standard and most popular technique, a needle driver is used to drive a curved needle attached to a suture through the fascia on either side of the port site. This method is challenging due to limited visualization through a small hole. In addition, the contents of the abdominal cavity are at risk of injury since the needle is driven through the fascia blindly. Some physicians use protection devices such as a groove director, but these do not guarantee an adequate bite of fascia necessary for a strong closure. For this reason the Carter Thompson system was developed which uses a standardized needle director to ensure adequate bites of fascia for a strong closure. Unfortunately, this device requires direct visualization from the camera, requires removing the trocar and losing the pneumoperitoneum necessary for use and visualization, and requires multiple passes of needle to deliver the suture. This complicated system improved the chances of a good closure but took a lot longer to carry out due to the number of steps and technical skill required.

A number of attempts over the past 20 years have been made to develop a device to aid in closure of these port sites and address the inadequacies of current devices. Limited examples of prior art include devices disclosed in U.S. Pat. No. 6,743,241 entitled “Laparoscopic port site fascial closure device”, U.S. Pat. No. 5,364,408 entitled “Endoscopic suture system”, U.S. Pat. No. 5,374,275 entitled “Surgical suturing device and method of use”, and U.S. Pat. No. 6,562,052 entitled “Suturing device and method”. These devices as described prove to be inadequate for fascial closure, primarily due the fact they are unable to obtain adequate tissue adjacent to the defect to provide a strong closure, they are not able to maintain pneumoperitoneum needed for appropriate visualization, they require multiple iterations of placing the instrument through the defect and removing the instrument, and they don't adequately protect either the vital structures in the vicinity of the tissue or the healthcare provider from risk of injury. As can be seen there is a need for a more reliable, safe and quick method for fascial closure.

### BRIEF SUMMARY OF THE INVENTION

This invention is an improved system to facilitate the closure of tissue defects created by the insertion of a trocar, sheath, or by any other suitable cause. While detail will be provided to aid in the enablement of the device for closure of laparoscopic fascial port sites, this should not limit the disclosure. Any suitable system or method that functions to close a tissue defect in the body may alternatively be used and should be considered within the scope of this disclosure.

The preferred embodiment of the instrument is an elongated device of appropriate diameter that can easily be inserted through the conduit used to create the tissue defect or the tissue defect itself (fascial opening in this case). The device is designed to be in profile where all the features are contained within the diameter of the device. If inserted through the conduit, the conduit (trocar) can then be slid over the entire instrument to remove the conduit without losing pneumoperitoneum.

The instrument has two opposing pivoting extensions at the distal end of the device. When the physician receives the device, it will be in its rest position where the two opposing pivoting extensions are at an angle away from the body of the device. Before placing the device through the conduit or tissue defect, the physician will use the pivoting extension actuator to bring the pivoting extensions in profile with the body of the device. This will allow the device to be placed into the conduit or tissue defect. The pivoting extension actuator is then released. The device is advanced through the conduit or tissue defect until the pivoting extensions pass the inner most tissue layer needing closure adjacent to the tissue defect or the tip of the conduit. At this point the pivoting extensions will revert back to the rest position. An indicator on the body of the device will alert the physician when this happens. This safety mechanism will ensure the device is not advanced too far through the tissue defect, essentially protecting all the vital structures beyond the tissue defect.

If the device was placed through the conduit, the conduit can then be removed by sliding it over the device. The device is then brought up so that the distal ends of the pivoting extensions appose the innermost tissue layer. As the physician pulls up on the device, the pivoting extensions open by sliding along the innermost tissue layer and sweeping away any vital structures that may be close to the instrument. Alternatively the pivoting extensions can be opened using the collapsible handle connected to the pivoting extension actuator. An indicator on the device alerts the physician that the pivoting extensions are open in their final actuated position. The pivoting extensions are then locked into position stabilizing the device to ensure proper function.

The physician then uses a needle actuator to drive two diametrically opposed flexible nitinol needles housed in the shaft of the instrument out of the ports on the body of the device. The flexible needles are driven in opposite directions through the tissue layers the physician is interested in closing, towards the distal ends of the pivoting extensions. The flexible needles are driven out at a predetermined height and angle to ensure that an adequate amount of tissue is used to close the tissue defect. The flexible needles need to be flexible enough to be manipulated within the body of the device and driven to an adequate distance away from the body of the device to obtain enough tissue for a strong closure. The flexible needles are also configured to have the appropriate stiffness to pierce through the tissue of interest without buckling. The flexible needles will be driven into the coupling channel to be received in the pivoting extensions by a set of couplers attached to the same suture that would be used otherwise to close the port sites. The distal ends of the flexible needles are configured to mate with the couplers and pull the suture back through the tissue adjacent to the tissue defect.

The physician then releases the lock on the pivoting extensions and uses the pivoting extension actuator to bring the pivoting extensions back into profile with the body. The device is then pulled out of the tissue defect, leaving a looped suture through the tissue adjacent to the tissue defect. The physician can then tie the sutures as he or she would normally do in the standard procedure. At any time, the physician can reposition or remove the instrument without placing the sutures. The procedure may be repeated as many times as the physician considers necessary (usually 1 to close the 10 or 12 mm fascial ports and 2 or 3 to close the 25 mm fascial port) after replacing a cartridge on the distal end of the device.

A number of features have been incorporated into the device to ensure the safety of the patient, the physician and the reliability of the device. The device guarantees a good closure by consistently obtaining a sufficient amount of tissue adjacent to the tissue defect every time due to the use of flexible nitinol needles and the geometry of the needle tract. The pivoting extensions have been designed to ensure that all intra-abdominal contents have been swept out of the path of the flexible needles. The pivoting extensions also protect all the contents during the deployment of the flexible needles. To prevent accidental deployments of the flexible needles, the pivoting extensions have to be in locked into position prior to needle deployment. The safety features designed into the device allow it to be used with and without visualization or pneumoperitoneum. Finally, the device was designed so that it would automatically place a suture in less than 30 seconds in 1-2-3 step fashion.

### BRIEF DESCRIPTION OF THE FIGURES

FIG. 1A is an isometric view of a preferred embodiment of the suturing apparatus to close tissue defects described previously.

FIG. 1B is an isometric, cross-sectional view of the apparatus depicted in FIG. 1A.

FIG. 1C is a bottom view depicting the profile of the apparatus. FIG. 1D is a side view of the tissue defect with all the appropriate layers and the conduit inserted through the defect.

FIG. 2A is a front view of the distal end of the apparatus depicted in FIGS. 1A and 1B where the pivoting extensions are moving into their first position in preparation for entry into a tissue defect.

FIG. 2B is a front view of the distal end of the apparatus depicted in FIGS. 1A and 1B in which the distal end of the apparatus is inserted into the tissue defect and the pivoting extensions are restrained in position one by the surrounding tissue defect.

FIG. 2C is a front view of the distal end of the apparatus depicted in FIGS. 1A and 1B in which the pivoting extensions have returned to their resting position after the distal portion of the apparatus has past beyond the innermost tissue layer adjacent to the tissue defect and continue to pivot away from the body of the apparatus consequently sweeping away vital structures that reside in the vicinity of the defect as the apparatus is pulled upward from the defect.

FIG. 2D is a front view of the distal end of the apparatus depicted in FIGS. 1A and 1B in which the pivoting extensions continue to pivot away from the body of the apparatus while sweeping away vital structures that reside in the vicinity of the tissue defect as the apparatus is pulled upward through the defect.

FIG. 2E is a front view of the distal end of the apparatus depicted in FIGS. 1A and 1B in which the pivoting extensions have been stabilized in their position three such that the anterior of the pivoting extensions are in complete apposition with the innermost layer of tissue adjacent to the tissue defect.

FIG. 3A is a front view of the distal end of the apparatus depicted in FIGS. 1A and 1B in which the flexible needles have exited the body of the apparatus at an angle to the central axis of the body of the apparatus penetrating the tissue layers adjacent to the tissue defect.

FIG. 3B is an isometric view of the distal end of the apparatus depicted in FIGS. 1A and 1B depicting extension the flexible needles entering the coupler channels of the pivoting extensions, thereby engaging the couplers and the opposing ends of the suture.

FIG. 3C is an isometric, partial cross-sectional view of the distal end of the apparatus depicted in FIGS. 1A and 1B depicting the coupler which resides in the coupler channel of the pivoting extensions mated to the distal end of the flexible needle and the opposing ends of the suture.

FIG. 3D is a front view of the distal end of the apparatus depicted in FIGS. 1A and 1B in which the flexible needles have engaged the opposing couplers and are returning toward the body of the apparatus while pulling the opposing ends of the suture through the tissue layer adjacent to the tissue defect.

FIG. 3E is a front, partial cross-sectional view of the distal end of the apparatus depicted in FIGS. 1A and 1B in which the flexible needles have fully receded into the needle tracts, bringing the couplers into the ports of the body of the apparatus.

FIG. 4A is a front, cross-sectional view of the distal end of the apparatus depicted in FIGS. 1A and 1B depicting the pivoting extension stabilizer as the apparatus is pulled upward while the pivoting extensions sweep along the tissue layer of the defect.

FIG. 4B is a front, cross-sectional view of the distal end of the apparatus depicted in FIGS. 1A and 1B depicting the pivoting extension stabilizer engaging the paired pivoting extensions restraining them in position three.

FIG. 5 is a front, cross-sectional view of the distal end of the apparatus depicted in FIGS. 1A and 1B depicting the path in the needle tract that is followed by the flexible needle as it moves towards the coupler channel of the pivoting extension.

### DETAILED DESCRIPTION OF THE PREFERRED EMBODIMENTS

The detailed description set forth below is intended as a description of the presently preferred embodiments of the invention and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

This invention is an improved system to facilitate the closure of tissue defects created by the insertion of a trocar, sheath, or by any other suitable cause. While detail will be provided to aid in the enablement of the device for closure of laparoscopic fascial port sites, this should not limit the disclosure. Any suitable system or method that functions to close a tissue defect in the body may alternatively be used and should be considered within the scope of this disclosure.

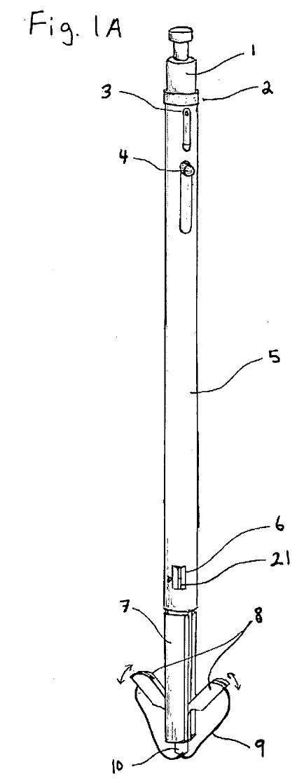

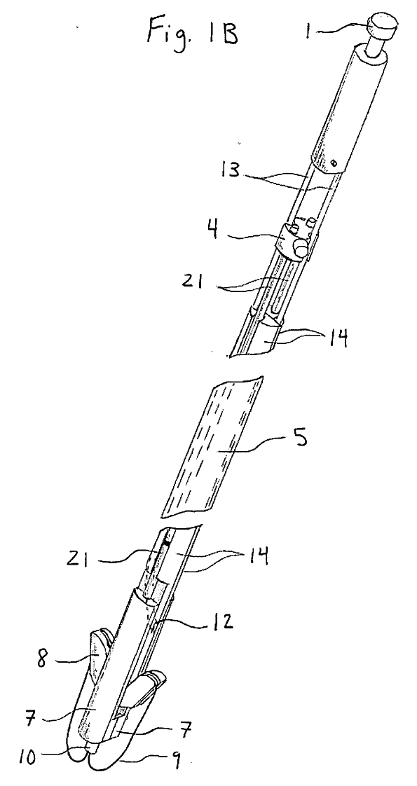

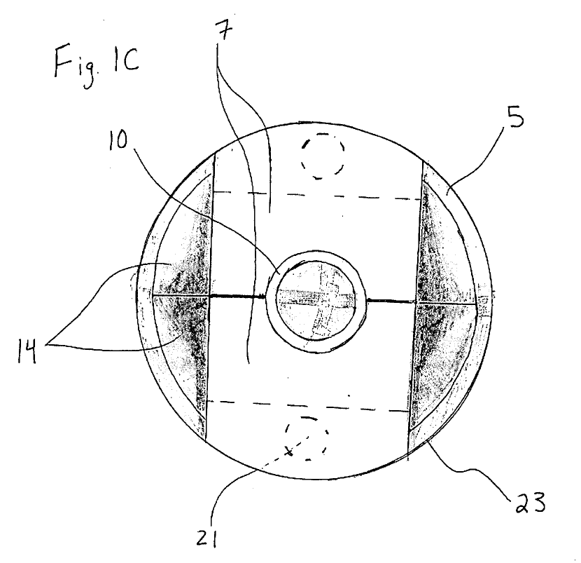

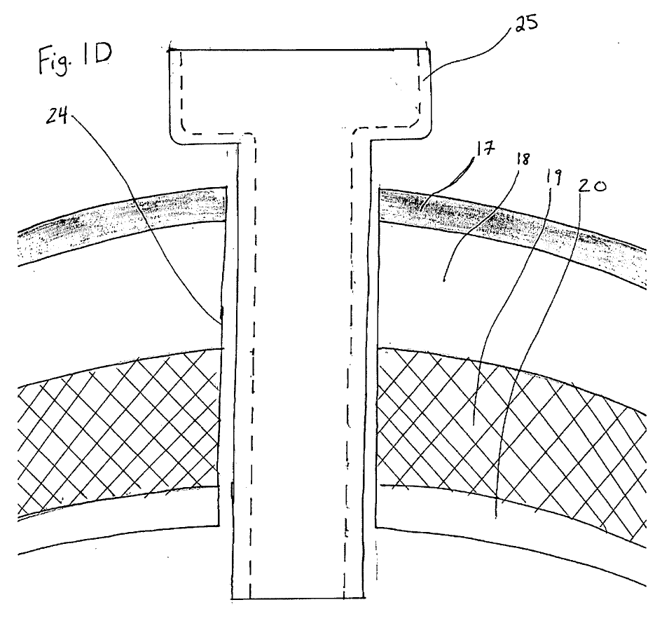

FIG. 1D represents the preferred tissue defect to be closed by the instrument. This comprises the tissue defect 24 with adjacent tissue comprising the innermost layer 20, a muscle layer 19, fat layer 18, and skin 17. The innermost layer 20 is targeted to be the most important layer in closure and in this explanation represents fascia. FIG. 1D also shows a conduit 25 placed through the tissue defect 24. Again in this example the conduit represents a trocar used in laparoscopy.

The preferred embodiment of the instrument shown in FIG. 1A, FIG. 1B, FIG. 1C is an elongated body 5 of appropriate diameter 23 that can easily be inserted through the conduit 25 shown in FIG. 1D used to create the tissue defect 24 also shown in FIG. 1D or the tissue defect 24 itself (fascial opening in this case).

Shown in FIGS. 1A and 1B, the instrument comprises a needle actuator 1 with a proximal and distal end where the distal end is rigidly fixated to a multiple of flexible needles 13. The distal end of the needle actuator 1 is housed in the body 5 of the instrument while the proximal end is outside the body 5 of the instrument. The needle actuator 1 moves in a proximal to distal and a distal to proximal direction through the body 5 to drive the flexible needles 13 through the needle tract 14 to ensure the proper direction and target for the flexible needles 13.

The pivoting extension actuator 7 having a proximal and distal end is free to slide over the distal end of the needle tract 14 and is rigidly connected to a collapsible handle 4 via a push rod 21 and houses the pivoting extensions 8 distal to the body 5. The pivoting extension actuator 7 is attached to the pivoting extensions 8 and used to actuate the pivoting extensions 8 by bringing said pivoting extensions 8 into contact with the tapered distal end of the needle tracts 14. A locking mechanism 2 shown in FIG. 1A at the proximal end of body 5 is shown to lock the pivoting extension actuator 7 in the actuated position for the pivoting extensions 8 that are attached to the distal end of the pivoting extension actuator 7. A needle limiter 3 is also shown in FIG. 1A on the distal aspect of the needle actuator 1 provided to prevent the couplers 22 from disengaging the flexible needles 13 when the needle actuator 1 moves from a distal to proximal direction to retract the flexible needles 13 into the ports 12 of the needle tracts 14.

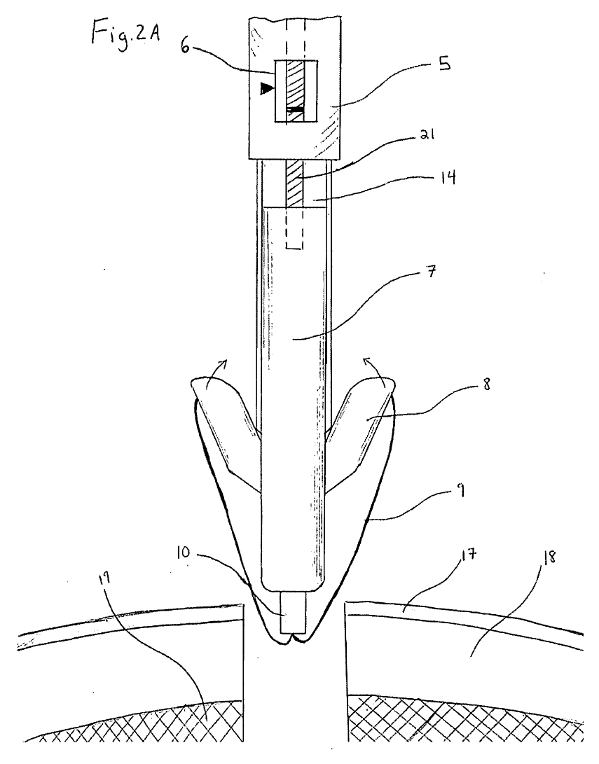

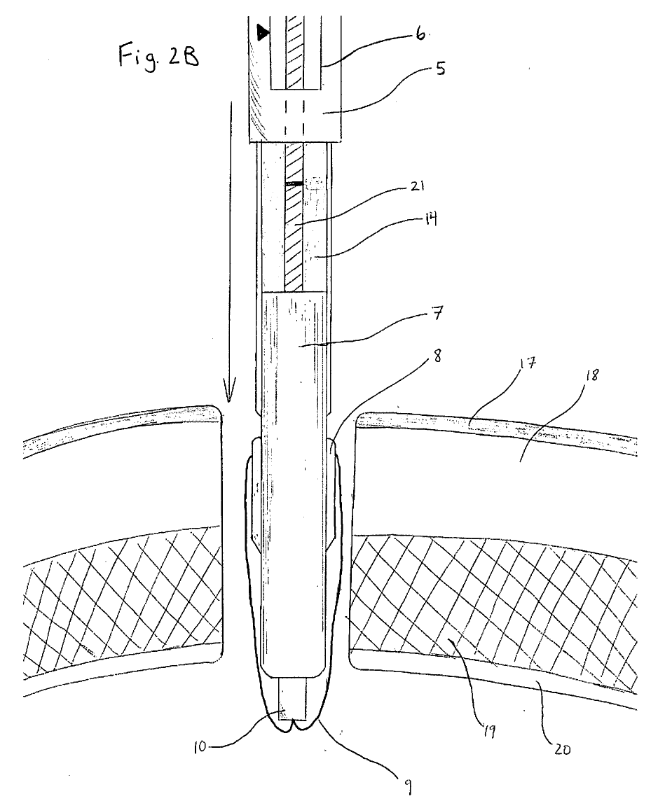

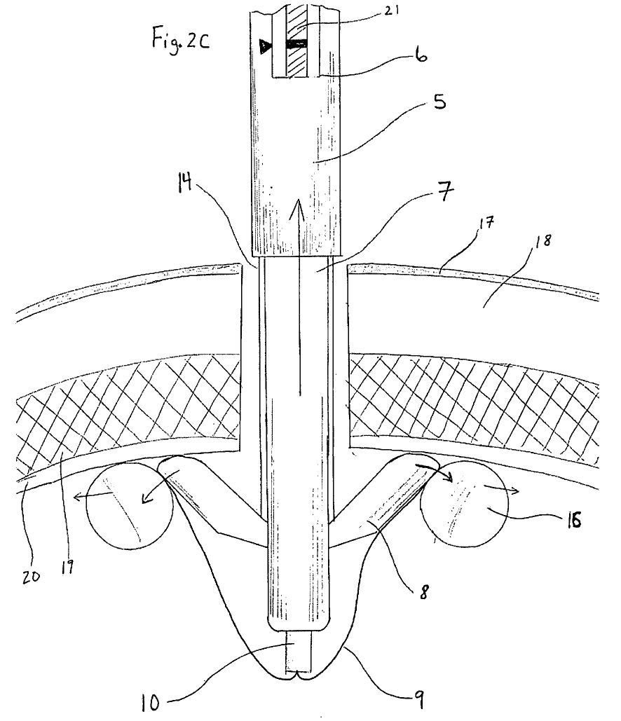

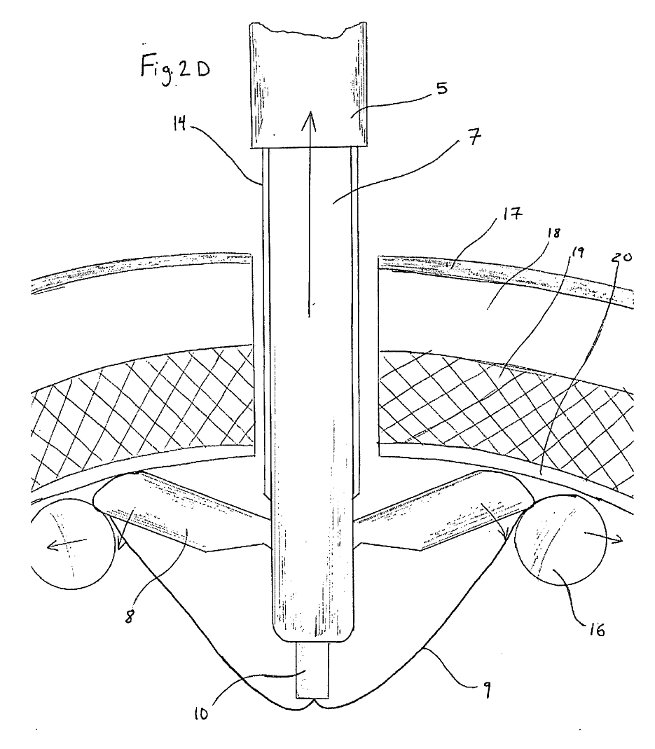

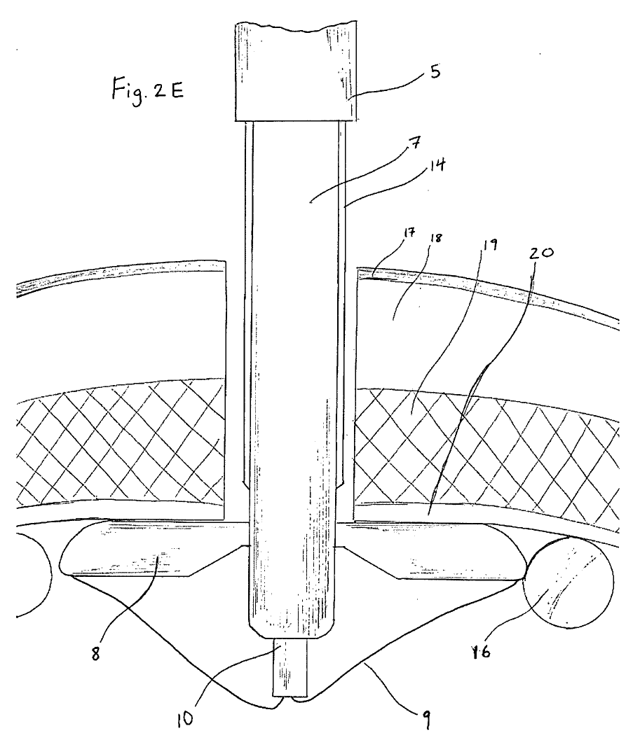

Also shown in FIG. 1A is the depth indicator 6 which alerts the physician when the pivoting extensions 8 move from position one where the pivoting extensions 8 are in line with the body 5 as shown in FIG. 2B to position two where the pivoting extension 8 move to an angle away from the body 5 as shown in FIG. 2C upon crossing the innermost tissue layer 20 which is adjacent to the tissue defect 24.

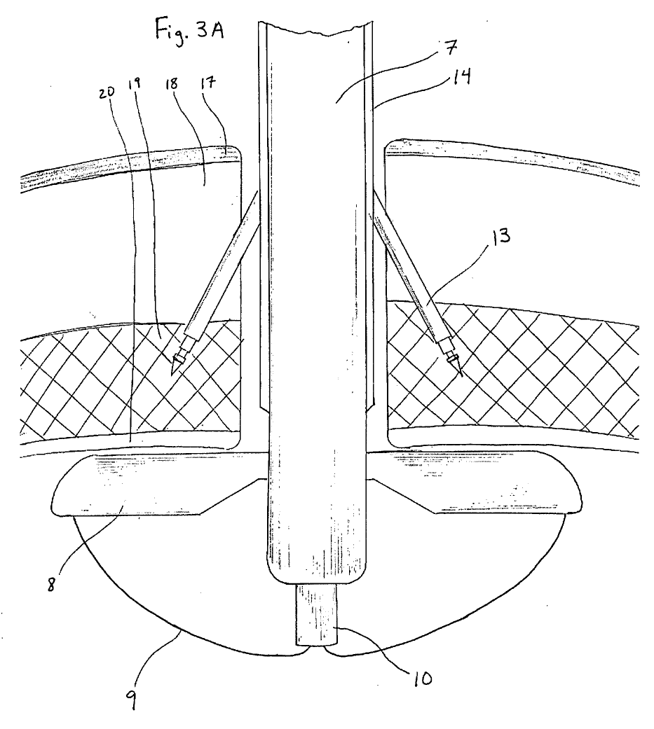

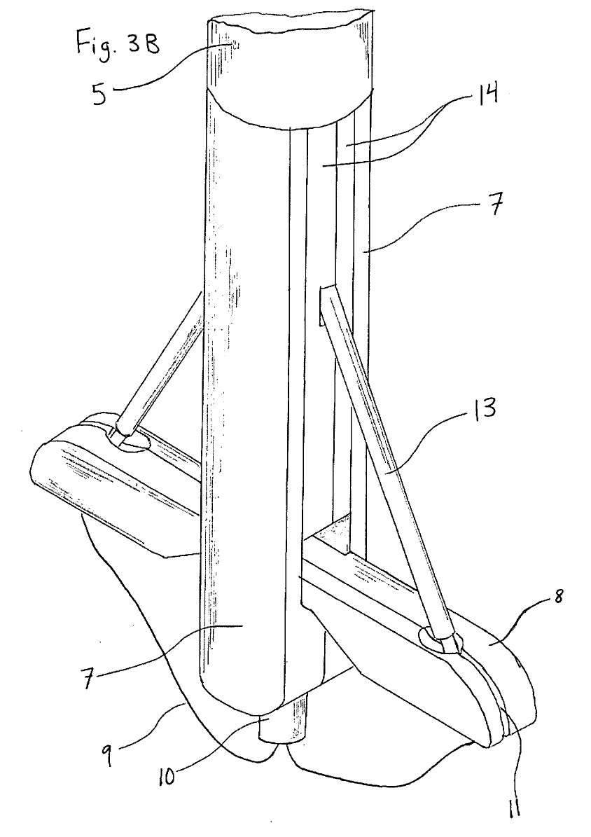

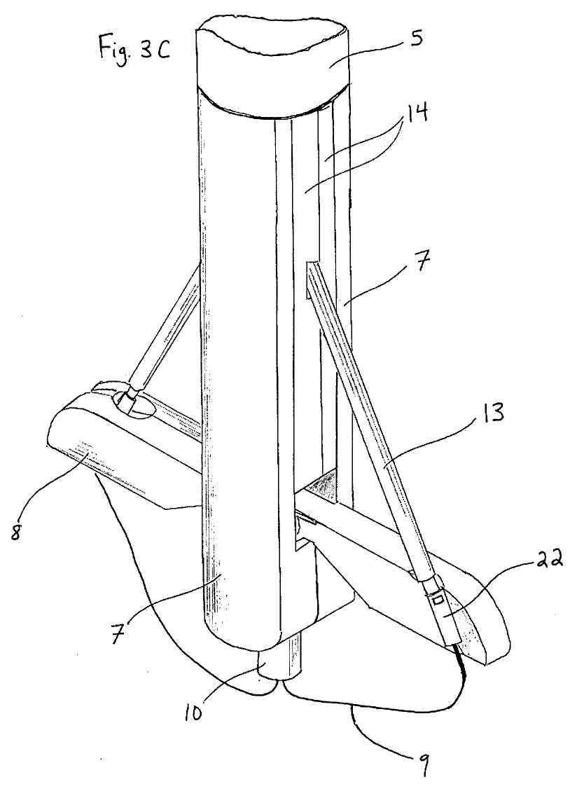

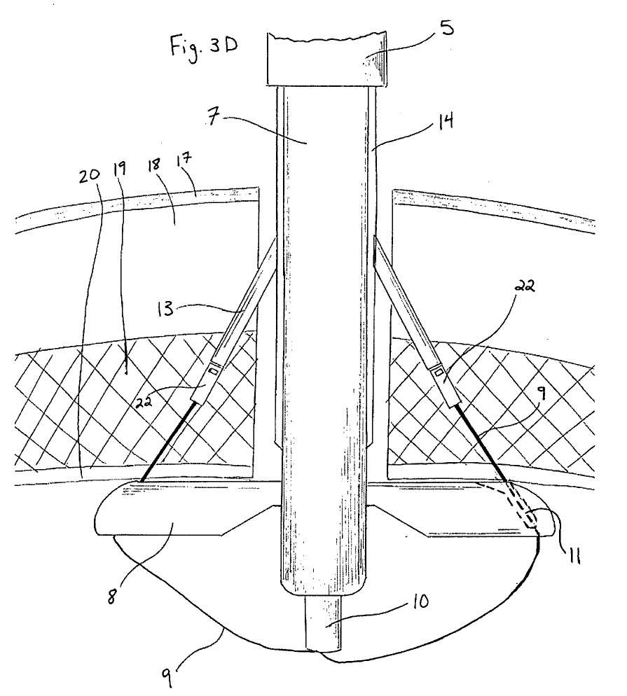

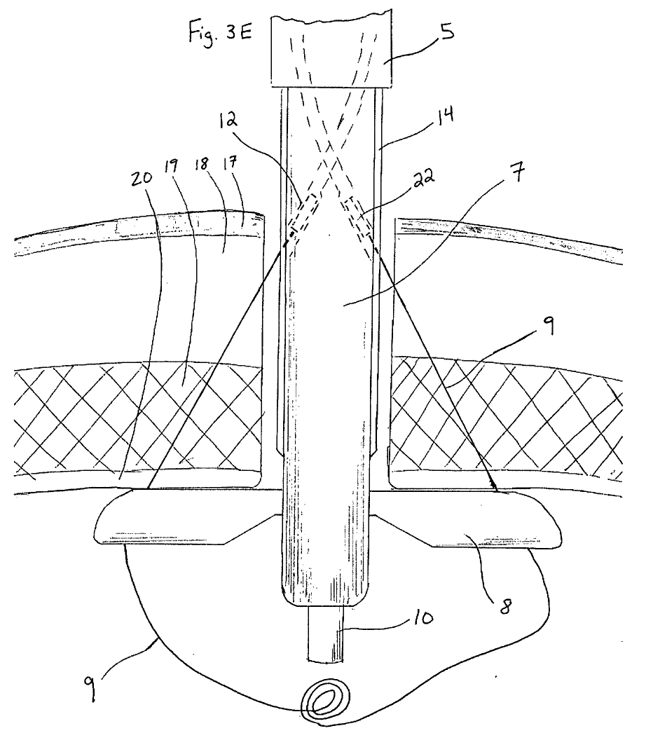

Also shown in FIG. 1A is the suture storage compartment 10 which houses the suture 9 whose distal ends are attached to the distal ends of couplers 22 which reside in the coupler channels 11 shown in FIG. 3D at the distal end of the pivoting extensions 8.

The device is designed to be in profile as shown in FIG. 1C where all the features are contained within the appropriate diameter 23 of the device when the pivoting extensions 8 are in their first position. If inserted through the conduit 25, the conduit 25 (trocar) can then be slid over the instrument to remove the conduit 25 without losing pneumoperitoneum.

FIGS. 2A, 2B, and 2C show the insertion of the device through the tissue defect 24 and/or conduit 25. The instrument has two opposing pivoting extensions 8 at the distal end of the device. When the physician receives the device, the pivoting extensions 8 will be in position two or its rest position where the two opposing pivoting extensions 8 are at an angle away from the body 5 of the device as show in FIG. 2A. Before placing the device through the conduit 25 or tissue defect 24, the physician will use collapsible handle 4 to engage the pivoting extension actuator 7 to bring the pivoting extensions 8 in profile with the body 5 of the device as show in FIG. 2B. This will allow the device to be placed into the conduit 25 or tissue defect 24. The collapsible handle 4 is then released. The device is advanced through the conduit 25 or tissue defect 24 until the pivoting extensions 8 pass the inner most tissue layer 20 needing closure adjacent to the tissue defect 24 or the tip of the conduit 25. At this point the pivoting extensions 8 will revert back to the rest position or position two as show in FIG. 2C. A depth indicator 6 on the body 5 of the device will alert the physician when this happens. This safety mechanism will ensure the device is not advanced too far through the tissue defect 5, essentially protecting all the vital structures 16 beyond the tissue defect 24.

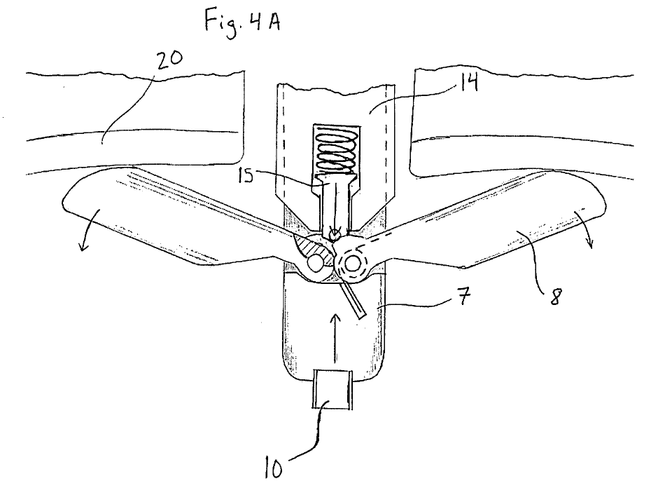

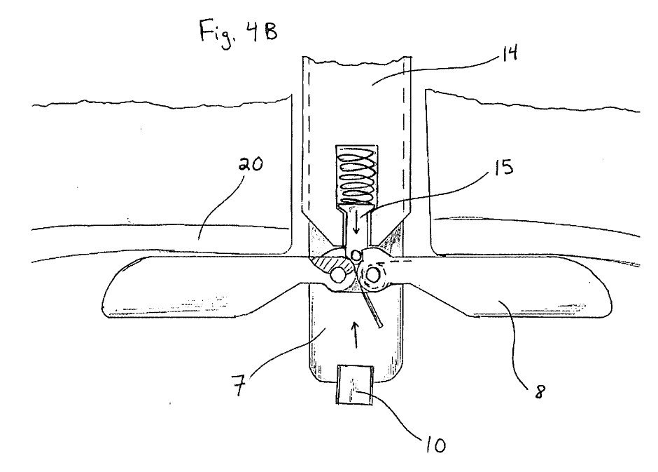

If the device was placed through the conduit 25, the conduit 25 can then be removed by sliding it over the device. As shown in FIG. 2C the device is then brought up so that the distal ends of the pivoting extensions 8 appose the innermost tissue layer 20. As the physician pulls up on the device, the pivoting extensions 8 open sliding along the innermost tissue layer 20 and sweeping away any vital structures 16 that may be close to the instrument as shown in FIG. 2D and FIG. 4A. Alternatively the pivoting extensions 8 can be opened using the pivoting extension actuator 7. An indicator 6 on the device alerts the physician that the pivoting extensions 8 are open in their final actuated position or position three as shown in FIG. 2E and FIG. 4B. The pivoting extensions 8 then lock into position stabilizing the device to ensure proper function.

FIGS. 4A and 4B show the pivoting extensions 8 moving from position two to position three and locking into place to stabilize the device. This is done with a pivoting extension stabilizer 15 which drops and apposes the proximal end of the pivoting extensions 8.

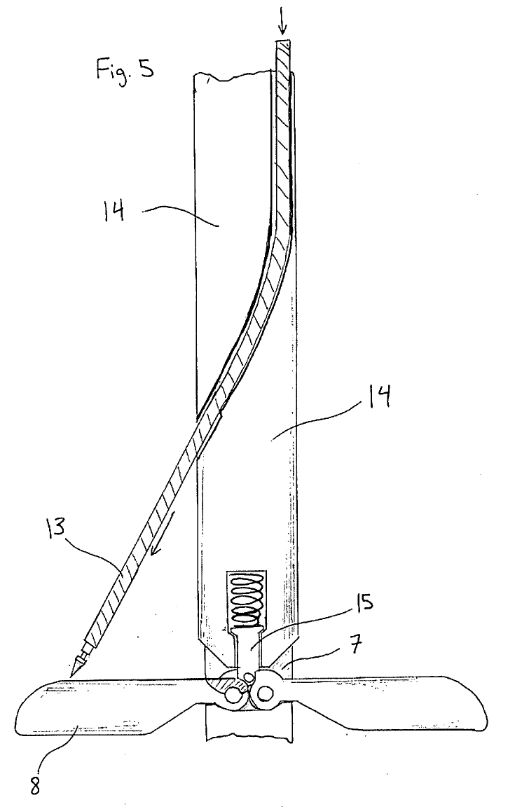

Once the pivoting extensions 8 are in position three, as shown in FIGS. 3A, 3B, 3C, 3D, 3E and FIG. 5, the physician then uses a needle actuator 1 to drive two diametrically apposed flexible nitinol needles 13 via the needle tract 14 housed in the body 5 of the instrument out of the ports 12 on the needle tracts 14. The flexible needles 13 are driven in opposite directions through the tissue layer 20 the physician is interested in closing, towards the distal ends of the pivoting extensions 8. The flexible needles 13 are driven out at a predetermined height and angle to ensure that an adequate amount of tissue 20 is used to close the tissue defect 24. The flexible needles 13 need to be flexible enough to be manipulated within the body 5 of the device and driven to an adequate distance away from the body 5 of the device to obtain enough tissue 20 for a strong closure. The flexible needles 13 are also configured to have the appropriate stiffness to pierce through the tissue 20 of interest without buckling.

FIGS. 3C, 3D and 3E show the flexible needles 13 being driven into the coupling channel 11 to be received in the pivoting extensions 8 by a set of couplers 22 attached to the same suture 9 that would be used otherwise to close the tissue defect 24. The distal ends of the flexible needles 13 are configured to mate with the couplers 22 and pull the suture 9 back through the tissue 20 adjacent to the tissue defect 24.

FIG. 3E shows the flexible needles 13 engaged to the couplers 22 attached to the suture 9 retracted into the ports 12 of the body 5 to ensure the profile 23 of the device is maintained upon removal from the tissue defect 24.

The physician then releases the locking mechanism 2 on the pivoting extensions 8 and uses the collapsible handle 4 to engage the pivoting extension actuator 7 to bring the pivoting extensions 8 back into profile with the body 5. The device is then pulled out of the tissue defect 24, leaving a looped suture 9 through the tissue 20 adjacent to the tissue defect 24. The physician can then tie the suture 9 as he or she would normally do on the standard procedure. At any time, the physician can reposition or remove the instrument without placing the sutures 9. The procedure may be repeated as many times as the physician considers necessary (usually 1 to close the 10 or 12 mm fascial ports and 2 or 3 to close the mm fascial port) after replacing a cartridge on the distal end of the device. A number of features have been designed into the device to ensure the safety of the patient and the physician and the reliability of the device. The device guarantees a good closure by consistently obtaining a predetermined amount of tissue 20 adjacent to the tissue defect 24 every time due to the use of flexible nitinol needles 13 and the geometry of the needle tract 14. The pivoting extensions 8 have been designed to ensure that all intra-abdominal contents 16 have been swept out of the path of the flexible needles 13. The pivoting extensions 8 also protect all the vital contents 16 during the deployment of the flexible needles 13. Enough safety features have been designed into the device, that it can be used with and without visualization or pneumoperitoneum. Finally, the device was designed so that it would automatically place a suture 9 in less than 30 seconds in 1-2-3 step fashion.

As a person skilled in the art will recognize from the previous descriptions and from the figures, modifications and changes can be made to the preferred embodiments of the invention without departing from the scope of this invention.

What is claimed is:

1. A suture applicator for assisting in the closure of tissue defects by apposing tissue adjacent to said tissue defects in a patient comprising:
(1) a slender cylindrical body configured to be introduced through the defect and or defect conduit with a proximal and distal end of sufficient length such that the proximal end is outside the body and the distal end is distal to the innermost layer of tissue adjacent to the tissue defect;
(2) a first and second diametrically opposed pivoting extensions at the distal end of said cylindrical body having a distal end and a proximal end and an anterior surface and a posterior surface;
(3) said extensions having a coupler channel on the distal end to mount the opposing ends of the suture in said first and second extensions such that said distal ends or coupler channels are able to rotate towards said body to a first position which is in line with said body and away from said body into a second position and third position where the third position is perpendicular to said body and the second position is between the first and third positions;
(4) a first and second flexible needles having a distal and proximal ends which resides in a needle channel in said body configured to allow said flexible needles to move from a proximal to distal direction and distal to proximal direction such that said flexible needles exit from diametrically opposed ports on said body at a height adjacent to said body outwards from said body at an angle towards said coupler channels in said pivoting extensions when in said second or third position, such that the distal ends of said flexible needles traverse adequate tissue adjacent to the tissue defect to engage the opposing ends of the suture residing in said coupler channels to allow adequate closure of the said tissue defects;
(5) a needle actuator having a proximal and distal end in line with the proximal end of said body rigidly fixated to the proximal end of said flexible needles to allow the proximal to distal movement of said flexible needles towards said coupler channel to engage the end portion of the suture and to allow the distal to proximal movement of said flexible needles to draw said suture through the tissue adjacent to the tissue defect;
(6) a suture storage on the distal end of said body configured to allow the suture to unravel after said flexible needles engage couplers attached to said suture and retract into said ports.

2. The applicator as defined in claim 1 where position two is the resting position and said body contains a pivoting extension actuator with a distal end and a proximal end wherein the distal end engages said pivoting extensions to rotate said pivoting extensions from said position two to said position one by proximal to distal movement of said pivoting extension actuator in relation to said body or from said position two to said position three by distal to proximal movement of said pivoting extension actuator in relation to said body.

3. The applicator as defined in claim 2 wherein said pivoting extension actuator stabilizes said pivoting extensions and said body by locking said pivoting extensions in said position three with a stabilizing mechanism at the distal end of said pivoting extension actuator comprising a moving element that engages the proximal end of said pivoting extension when said pivoting extension is in said position three such that said stabilizing mechanism can be fixed with a locking mechanism at the proximal end of said pivoting extension actuator comprising a lock and release element to fixate the proximal end of the pivoting extension actuator.

4. The applicator as defined in claim 1 wherein said pivoting extensions are configured such that on moving said apparatus from a distal to proximal direction, said pivoting extensions oppose the inner most layer of tissue adjacent to the tissue defect and move from said position two to said position three sliding along the tissue adjacent to said tissue defect sweeping any vital structures out of harms way such that said anterior surface of said pivoting extensions completely or partially apposes the inner most layer of tissue adjacent to said tissue defect.

5. The applicator as defined in claim 1 wherein said body has a depth indicator located between the proximal and distal ends of said body such that the depth indicator indicates when said pivoting extensions rotate from said position one to said position two or from said position two to said position one allowing said apparatus to appose the innermost layer of tissue adjacent to said tissue defect without harming any vital structures on traversing said tissue defect.

6. The applicator as defined in claim 1 where in said pivoting extensions contain couplers located in said coupler channels where said couplers having a distal end and a proximal end where said couplers are configured to engage the distal end of said flexible needles at the proximal end of said coupler and attach suture to the distal end of said coupler.

7. The applicator as defined in claim 1 wherein said body contains diametrically apposed said ports between the proximal and distal ends of said body where said ports are configured in a manner to allow said flexible needles to exit towards said coupler channels to engage said couplers and retract said flexible needles and attached couplers back into said ports such that said coupler and said flexible needle are completely within said body.

8. The applicator as defined in claim 1 wherein said applicator is in profile allowing the passage of said apparatus through a conduit and the passage of a conduit over entire said apparatus.

9. A suture applicator for assisting in the closure of tissue defects by apposing tissue adjacent to said tissue defects such that said applicator is in profile allowing the passage of said apparatus through a conduit and the passage of a conduit over entire said apparatus in a patient comprising:
(1) a slender cylindrical body configured to be introduced through the defect and or defect conduit with a proximal and distal end of sufficient length such that the proximal end is outside the body and the distal end is distal to the innermost layer of the tissue defect;
(2) a first and second diametrically opposed pivoting extensions at the distal end of said cylindrical body having a distal end and a proximal end and an anterior surface and a posterior surface;
(3) said extensions having a coupler channel on the distal end to mount the opposing ends of the suture in said first and second extensions such that said distal ends or coupler channels are able to rotate towards said body to a first position which is in line with said body and away from said body into a second position and third position where the third position is perpendicular to said body and the second position is between the first and third positions;
(4) a first and second flexible needles having a distal and proximal ends which resides in a needle channel in said body configured to allow said flexible needles to move from a proximal to distal direction and distal to proximal direction such that said flexible needles exit from diametrically opposed ports on said body at a height adjacent to said body outwards from said body at an angle towards said coupler channels in said pivoting extensions when in said second or third position, such that the distal ends of said flexible needles traverse adequate tissue adjacent to the tissue defect to engage the opposing ends of the suture residing in said coupler channels to allow adequate closure of the said tissue defects;
(5) a needle actuator having a proximal and distal end in line with the proximal end of said body rigidly fixated to the proximal end of said flexible needles to allow the proximal to distal movement of said flexible needles towards said coupler channel to engage the end portion of the suture and to allow the distal to proximal movement of said flexible needles to draw said suture through the tissue adjacent to the tissue defect;
(6) a suture storage on the distal end of said body configured to allow the suture to unravel after said flexible needles engage couplers attached to said suture and retract into said ports.

10. The applicator as defined in claim 9 where position two is the resting position and said body contains a pivoting extension actuator with a distal end and a proximal end wherein the distal end engages said pivoting extensions to rotate said pivoting extensions from said position two to said position one by proximal to distal movement of said pivoting extension actuator in relation to said body or from said position two to said position three by distal to proximal movement of said pivoting extension actuator in relation to said body.

11. The applicator as defined in claim 10 wherein said pivoting extension actuator stabilizes said pivoting extensions and said body by locking said pivoting extensions in said position three with a stabilizing mechanism at the distal end of said pivoting extension actuator comprising a moving element that engages the proximal end of said pivoting extension when said pivoting extension is in said position three such that said stabilizing mechanism can be fixed with a locking mechanism at the proximal end of said pivoting extension actuator comprising a lock and release element to fixate the proximal end of the pivoting extension actuator.

12. The applicator as defined in claim 9 wherein said pivoting extensions are configured such that on moving said apparatus from a distal to proximal direction, said pivoting extensions oppose the inner most layer of tissue adjacent to the tissue defect and move from said position two to said position three sliding along the tissue adjacent to said tissue defect sweeping any vital structures out of harms way such that said anterior surface of said pivoting extensions completely or partially apposes the inner most layer of tissue adjacent to said tissue defect.

13. The applicator as defined in claim 9 wherein said body has a depth indicator located between the proximal and distal ends of said body such that the depth indicator indicates when said pivoting extensions rotate from said position one to said position two or from said position two to said position one allowing said apparatus to appose the innermost layer of tissue adjacent to said tissue defect without harming any vital structures on traversing said tissue defect.

14. The applicator as defined in claim 9 where in said pivoting extensions contain couplers located in said coupler channels where said couplers having a distal end and a proximal end where said couplers are configured to engage the distal end of said flexible needles at the proximal end of said coupler and attach suture to the distal end of said coupler.

15. The applicator as defined in claim 9 wherein said body contains diametrically apposed said ports between the proximal and distal ends of said body where said ports are configured in a manner to allow said flexible needles to exit towards said coupler channels to engage said couplers and retract said flexible needles and attached couplers back into said ports such that said coupler and said flexible needle are completely within said body

16. A suture applicator for assisting in the closure of tissue defects by apposing tissue adjacent to said tissue defects such that said applicator is in profile allowing the passage of said apparatus through a conduit and the passage of a conduit over entire said apparatus in a patient comprising:
(1) a slender cylindrical body configured to be introduced through the defect and or defect conduit with a proximal and distal end of sufficient length such that the proximal end is outside the body and the distal end is distal to the innermost layer of the tissue defect;
(2) a first and second diametrically opposed pivoting extensions at the distal end of said cylindrical body having a distal end and a proximal end and an anterior surface and a posterior surface wherein said pivoting extensions are configured such that on moving said apparatus from a distal to proximal direction, said pivoting extensions oppose the inner most layer of tissue adjacent to the tissue defect and move from said position two to said position three sliding along the tissue adjacent to said tissue defect sweeping any vital structures out of harms way such that said anterior surface of said pivoting extensions completely or partially apposes the inner most layer of tissue adjacent to said tissue defect;
(3) said extensions having a coupler channel on the distal end to mount the opposing ends of the suture in said first and second extensions such that said distal ends or coupler channels are able to rotate towards said body to a first position which is in line with said body and away from said body into a second position and third position where the third position is perpendicular to said body and the second position is between the first and third positions;
(4) a first and second flexible needles having a distal and proximal ends which resides in a needle channel in said body configured to allow said flexible needles to move from a proximal to distal direction and distal to proximal direction such that said flexible needles exit from diametrically opposed ports on said body at a height adjacent to said body outwards from said body at an angle towards said coupler channels in said pivoting extensions when in said second or third position, such that the distal ends of said flexible needles traverse adequate tissue adjacent to the tissue defect to engage the opposing ends of the suture residing in said coupler channels to allow adequate closure of the said tissue defects;
(5) a needle actuator having a proximal and distal end in line with the proximal end of said body rigidly fixated to the proximal end of said flexible needles to allow the proximal to distal movement of said flexible needles towards said coupler channel to engage the end portion of the suture and to allow the distal to proximal movement of said flexible needles to draw said suture through the tissue adjacent to the tissue defect;
(6) a suture storage on the distal end of said body configured to allow the suture to unravel after said flexible needles engage couplers attached to said suture and retract into said ports wherein said distal end of said body containing said suture storage and suture with said couplers attached comprise a removable cartridge to allow said apparatus to place multiple sutures.

17. The applicator as defined in claim 16 where position two is the resting position and said body contains a pivoting extension actuator with a distal end and a proximal end wherein the distal end engages said pivoting extensions to rotate said pivoting extensions from said position two to said position one by proximal to distal movement of said pivoting extension actuator in relation to said body or from said position two to said position three by distal to proximal movement of said pivoting extension actuator in relation to said body.

18. The applicator as defined in claim 17 wherein said pivoting extension actuator stabilizes said pivoting extensions and said body by locking said pivoting extensions in said position three with a stabilizing mechanism at the distal end of said pivoting extension actuator comprising a moving element that engages the proximal end of said pivoting extension when said pivoting extension is in said position three such that said stabilizing mechanism can be fixed with a locking mechanism at the proximal end of said pivoting extension actuator comprising a lock and release element to fixate the proximal end of the pivoting extension actuator.

19. The applicator as defined in claim 16 wherein said body has a depth indicator located between the proximal and distal ends of said body such that the depth indicator indicates when said pivoting extensions rotate from said position one to said position two or from said position two to said position one allowing said apparatus to appose the innermost layer of tissue adjacent to said tissue defect without harming any vital structures on traversing said tissue defect.

20. The applicator as defined in claim 16 where in said pivoting extensions contain couplers located in said coupler channels where said couplers having a distal end and a proximal end where said couplers are configured to engage the distal end of said flexible needles at the proximal end of said coupler and attach suture to the distal end of said coupler.

21. The applicator as defined in claim 16 wherein said body contains diametrically apposed said ports between the proximal and distal ends of said body where said ports are configured in a manner to allow said flexible needles to exit towards said coupler channels to engage said couplers and retract said flexible needles and attached couplers back into said ports such that said coupler and said flexible needle are completely within said body.
